(19) United States
(12) Patent Application Publication
(10) Pub. No.: US 2022/0386851 A1
(43) Pub. Date: Dec. 8, 2022

(54) ENDOSCOPE STRUCTURE

(71) Applicant: SCIVITA MEDICAL TECHNOLOGY CO., LTD., Suzhou (CN)

(72) Inventor: Yi ZHANG, Suzhou (CN)

(73) Assignee: SCIVITA MEDICAL TECHNOLOGY CO., LTD., Suzhou (CN)

(21) Appl. No.: 17/776,260

(22) PCT Filed: Nov. 13, 2019
(86) PCT No.: PCT/CN2019/117816

§ 371 Date: May 12, 2022

(51) Int. Cl. A61B 1/005; A61B 1/04; A61B 1/00

(52) U.S. Cl. CPC A61B 1/0055; A61B 1/0057; A61B 1/04; A61B 1/00119

(57) ABSTRACT

The present invention relates to the field of medical devices and discloses an endoscope structure, which consists of a fixing ring, a connection tube and an operation tube connected in sequence, among which, the connection tube consists of braided layers, the connection tube can be bent, and the stiffness of the braided layers of the connection tube is inversely proportional to the maximum bending angle of the connection tube; the structure also consists of traction wires, the traction wires are fixed onto the fixing ring by the operation tube through the connection tube, and the connection tube will be bent by pulling the traction wires at the end of the operation tube. Main beneficial effects of the present invention are: the braided layers and traction wires are arranged inside the connection tube, so that the present invention has advantages of convenient connection, soft and controllable; the guide groove is arranged on the outer lateral walls of the support layers, the traction wires are respectively placed inside the guide groove, when the traction wires move, the guide groove plays a role of guiding and positioning to ensure that the traction wires only move along the length direction of it structure.

### TECHNICAL FIELD

The present invention relates to the field of medical devices, particularly an endoscope structure.

### BACKGROUND TECHNOLOGY

With the continuous development of technologies, medical endoscope has been widely adopted in the medical field, and endoscope is an important device to detect organ and tissue lesions.

An endoscope mainly consists of a connection tube, a probe connected with the connection tube and a driving mechanism which drives the probe to bend at required angles, the traction wires of the driving mechanism are connected with the operation end of the connection tube. The probe arranged at the end of the connection tube runs into the human body, images of the organs and tissues around the probe are collected and transmitted to an external display for medical personnel to observe.

Presently, two types of ureteroscope are used: rigid and flexible ureteroscope. Rigid ureteroscope has advantages of good directionality and easy operation and placement, but it can't bend and result in blind areas of view and operation; Flexible ureteroscope can prevent blind areas of view and operation, but it has disadvantages of soft connection tube structure, poor controllability, difficult operation, high price and easy to be damaged.

It is found through patent search that, the open literature CN106963331A discloses a bronchoscope. However, in such open scheme, the control, wire and the fixing seat are clamped and fixed through positioning plates, the device has many parts, a complicated structure and high installation and manufacturing costs; moreover, the stability of clamping and fixing is not high, so there is a hidden danger that the adjustable bending function of the connection tube may fail during use when the control wire separates from the fixing seat.

### SUMMARY

The objective of the present invention is to design an endoscope structure in order to solve above issues.

The technical scheme of the present invention to achieve above objective is, an endoscope structure, which consists of a fixing ring, a connection tube and an operation tube connected in sequence, among which, the connection tube consists of braided layers, the connection tube can be bent, and the stiffness of the braided layers of the connection tube is inversely proportional to the maximum bending angle of the connection tube;

The structure also consists of traction wires, the traction wires are fixed onto the fixing ring by the operation tube through the connection tube, and the connection tube will be bent by pulling the traction wires at the end of the operation tube.

The connection tube is a hollow tube composed of several layers of materials, the innermost layer of the connection tube is support layer, the support layers are integrally formed, the braided layers are arranged and fit onto the outer lateral walls of the support layers, and the braided layers are braided with the same or different braiding yarns.

The outer diameter of the connection tube is bigger than that of the fixing ring, a positioning step is formed at the junction between the guide head and the support layer, and the end of the reinforcing coat facing the connection tube contacts with the positioning step.

A guide groove corresponding to the traction wires is arranged on the outer wall of the support layer, the traction wires are placed and can slide freely in the guide groove, and the guide groove is symmetrically arranged along the central axes of the support layers.

The connection tube consists of at least two connected part with different stiffness, the part close to the operation tube is stiffer than the part close to the fixing ring, the stiffness of the parts can be controlled through changing the stiffness of the braided layers inside such parts, the support layers in different parts are made of the same flexible and deformable material, and the flexible and deformable material can be PTFE.

The number of the traction wires is at least two, one ends of the traction wires are fixed onto the lateral wall of the fixing ring, and the other ends extend outside the end of the operation tube along the wall of the connection tube, when one of the traction wires is pulled, the connection tube will bend toward the direction of the bended traction wire.

The connection tube also consists of sleeves made of nylon elastomer material, the sleeves are arranged and fit onto the outer walls of the braided layers, a guide head used to connect with the camera lens is arranged at the end of the fixing ring, and a notch convenient for fixed installation of the camera lens is arranged on the guide head.

The notch is preferably a U-shaped notch, a reinforcing coat of the same shape with the guide head is sleeved and arranged outside the guide head to enhance the firmness of the guide head.

A reinforcing part is arranged at one end of the reinforcing coat close to the sleeve, the reinforcing coat is partially embedded in the reinforcing part.

The beneficial effects are that, 1. The braided layers and traction wires are arranged inside the connection tube, so that the present invention has advantages of convenient connection, soft and controllable. Among which, the braided layers have certain bending firmness, so that the overall strength of the connection tube can be enhanced, and difficult connection due to soft tube can be avoided; directional bending of the connection tube can be controlled by pulling the traction wire, the camera probe arranged at the tip of the connection tube can be controlled to reach desired positions in accordance with the requirements, so that the present invention has advantages of good directionality and convenient control.

2. The guide groove is arranged on the outer lateral walls of the support layers, the traction wires are respectively placed inside the guide groove, when the traction wires move, the guide groove plays a role of guiding and positioning to ensure that the traction wires only move along the length direction of it structure, accurate control of overall bending direction can be realized by controlling the traction wires, and better overall stability can be achieved.

The present invention adopts an integral structure composed of a fixing ring, a connection tube and an operation tube, the connection tube mainly consists of support layers and braided layers, the material cost is low, the structure is simple, the overall manufacturing cost is low, as a disposable medical product, the present invention, can greatly reduce medical cost.

### DESCRIPTION OF THE DRAWINGS

FIG. 1 is a sectional view of the overall structure of the present invention;

FIG. 2 is an enlarged view of Part A as shown in FIG. 1;

FIG. 3 is a sectional view of Section B-B as shown in FIG. 1;

FIG. 4 is a structural view of the wire trough in the present prevention;

FIG. 5 is a structural view of the connection between the traction wires and the reinforcing coat in the present prevention;

FIG. 6 is an action state diagram of bending leftward and rightward of the present invention;

In Figures, 1. Connection tube; 11. Flexible part; 12. Transition part; 13. Handheld part; 2. Operation, tube; 3. Braided layer; 4. Sleeve; 41. Reinforcing part; 5. Support layer; 51. Guide head; 511. Notch: 52. Positioning step; 53. Wire trough; 6. Reinforcing coat; 7. Traction wire; 8. Fixing ring.

### DESCRIPTION OF EMBODIMENTS

To be clear, the original design intention of the present invention is to address following disadvantages of current medical endoscope, particularly renal endoscope: rigid ureteroscope is difficult to be bent and results in blind areas of view and operation, flexible ureteroscope is too soft, not conductive to operation, and is expensive; the present invention intends to provide a new medical endoscope structure with advantages of both rigid and flexible ureteroscope.

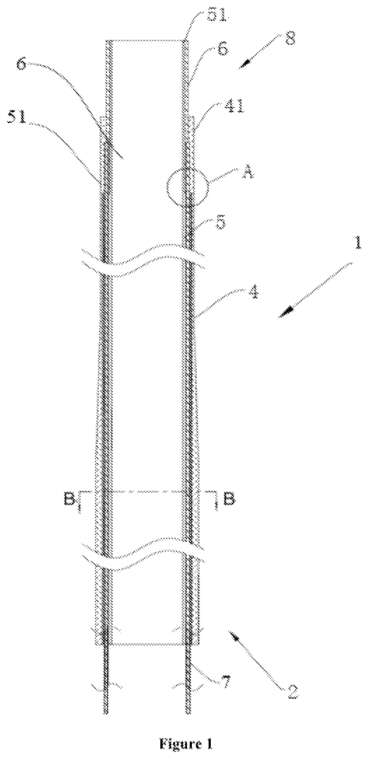

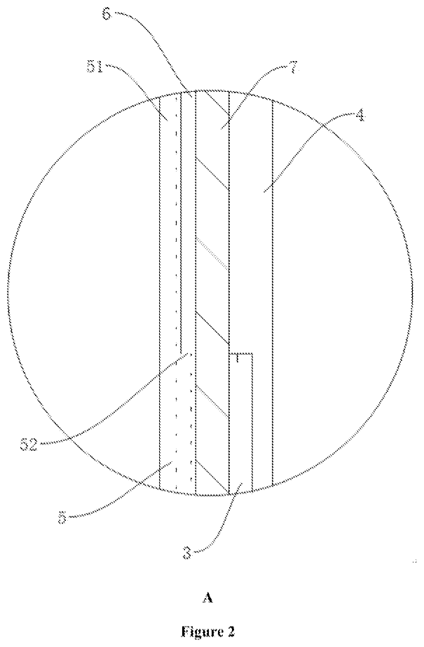

The present invention is described in detail below by combining FIGS. 1˜6.

The core mechanism of the endoscope structure defined in the present invention patent covers two aspects: a braided layer 3 is added among the layers of a connection tube 1 to make the connection tube 1 have certain stiffness and to prevent the flexible ureteroscope from being too soft; a traction wire 7 structure is designed in the tube, when the traction wires 7 are pulled, the traction wires 7 drive the head of the structure to bend, so the moving direction of the structure can be controlled at the tail of the structure (outside the human body), and the disadvantage of rigid ureteroscope being difficult to be bent can be avoided. The structure based on above two aspects is fully described below:

1. Description of the Structure and Principle of the Braided Layers 3.

As shown in FIG. 1, the basic structure of the present invention consists of the fixing ring 8, the connection tube 1 and the operation tube 2 connected in sequence, among which the connection tube 1 consists of the braided layers 3, and the connection tube 1 can be bent naturally.

The connection tube 1 is an intermediate tube connecting the camera, lens and the operation handle, and it is composed of several layers of materials; as one of such layers, the braided layers 3 can be braided and twined with the same or different braiding yarns, the stiffness of the corresponding braided layers 3 can be controlled by changing braiding material, braiding method and warp & weft density of braiding, the thicknesses of the braided layers 3 are preferably 0.025 mm˜0.03 mm. The braiding yarn of the braided layers 3 can be one or more of SS304, SS304V, SS304L, SS316 and SS316L, preferably SS304V. Braiding method can be braiding methods for twill weave structure, such as 1/2 [symbol] or 3/1 [symbol] , the warp & weft density of braiding can be directly measured with a density, the higher warp & weft density of braiding is, the better flexibility of the braided layer 3 will be.

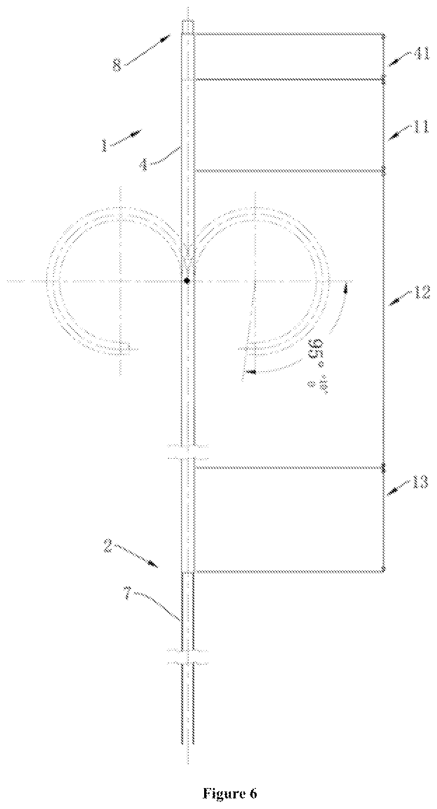

The connection tube 1 consists of at least two connected parts with different stiffness along the axial direction, each part is made of the same flexible and deformable material, the stiffness of such parts decrease in sequence from the operation tube 2 to the fixing ring 8; as a preferred embodiment, as shown in FIG. 6, the number of such part is set as 3, they are the handheld part 13, the transition part 12 and the flexible part 11 arranged in sequence along the direction from the operation tube 2 to the fixing ring 8. The warp & weft densities of the flexible part 11, the transition part 12 and the handheld part 13 gradually increase; the material of the sleeve 4 at the flexible part 11 is Pebax 6333 SA01, and its stiffness range is 35 HSD˜55 HSD; the material of the sleeve 4 at the transition part 12 is Pebax 7233 SA01, and its stiffness range is 55 HSD˜82 HSD; the material of the sleeve 4 at the handheld part 13 is PA12, and its stiffness range is 82 HSD˜85 HSD. Among which, Pebax 6333 SA01 and Pebax 7233 SA01 materials are the Pebax series products manufactured by French Arkema, and PA12 model is PA12 EMS TR-90.

Description of the structure and principle of the traction wires 7.

The traction wires 7 are fixed onto the fixing ring 8 by the operation tube 2 through the connection tube 1, and the connection tube 1 will be bent by pulling the traction wires 7 at the end of the operation tube 2.

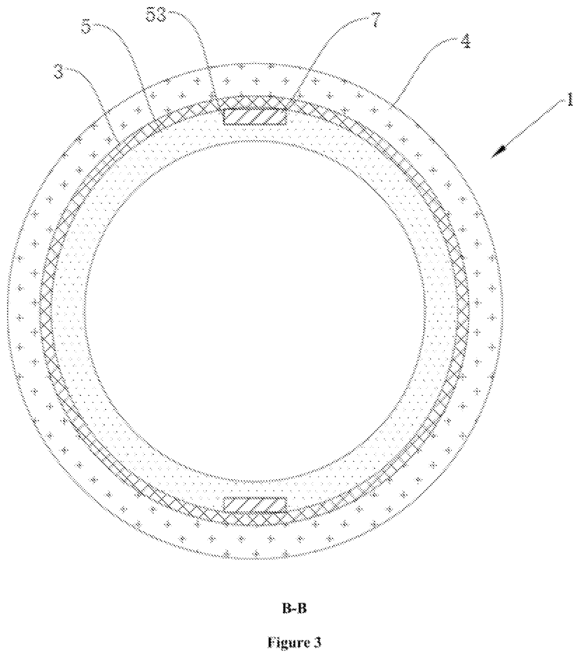

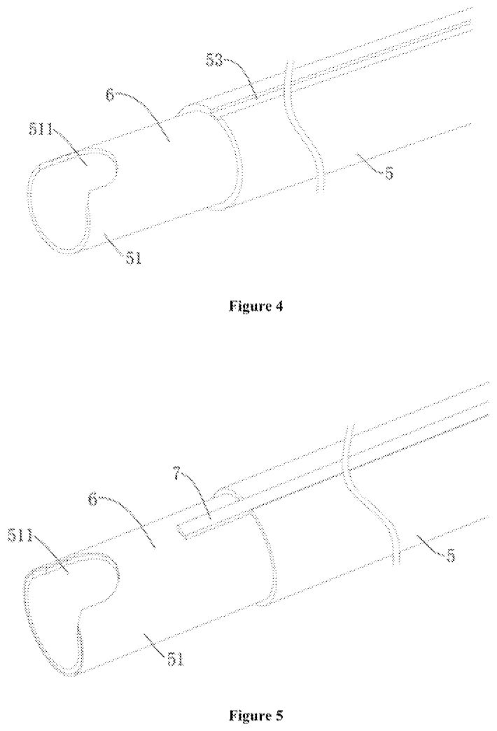

As shown in FIGS. 3˜5, the traction wires 7 controlling the bending degree of the connection tube 1 are symmetrically arranged on the lateral wall at one end of the reinforcing coat 6 close to the connection tube 1 along, the axis of such wall. During the surgery, the connection tube 1 and the imaging device are clamped onto the instrument since the positions of the reinforcing coat 6 and the support layers 5 are relatively fixed, when the traction wires 7 are pulled away from the fixing ring 8, the bending degree of the connection tube 1 can be adjusted, the operation is convenient and quick. Because the traction wires 7 need to be pulled and bent for a long time, so they are made of stainless steel, preferably SS304. If the traction wires 7 are connected with the sleeves 4, since the sleeves 4 are made of nylon elastomer material, when pulling the traction wires 7 to bend the connection tube 1, driving by force through the traction wires 7 is firstly needed, so that the traction wires 7 can't be reliably connected with the surfaces of the sleeves 4 and may easily fall off; if the traction wires 7 are fixed onto the inner lateral walls of the support layers 5 (FIG. 5), there will also be a disadvantage of the traction wires 7 not being reliably connected with PTFE material, meanwhile, the traction wires 7 may contact with the signal line of the imaging device, during pulling the traction wires 7, a poor contact between the imaging device and the signal line may be easily caused. Therefore, the traction wires 7 can only be fixed onto the outer lateral wall of the reinforcing coat 6, but the traction wires 7 can't directly contact with the braided layers 3, otherwise, unevenness may easily be caused when forming the sleeves 4, so as shown in FIG. 4, the wire troughs 53 are arranged on the support layers 5 along the length direction of the support layers 5, the traction wires 7 are embedded inside the wire troughs 53. and one ends of the traction wires 7 extend out of the connection tube 1. Since both the traction wires 7 and the reinforcing coat 6 are made of stainless steel, to guarantee the reliability of their connection, they are fixed by welding, finally, they are blocked by the sleeves 4, so that they will not contact with human body or the signal line. The traction wires 7 are flat wires or round wires stranded with several wires, in this embodiment, flat wires are adopted, with a cross-sectional dimension of 0.08 mm×0.3 mm, the dimensions of the traction wires 7 match with the wall thicknesses of the support layers 5, and the impact caused by the traction wires 7 on the stiffness of the entire connection tube 1 can't be ignored.

However, the braided layers 3 and the traction wires 7 do not constitute a complete technical scheme, in order to make the present invention more specific, further description is provided below:

The innermost layer of the connection tube 1 is the support layer 5, the support layers are integrally formed, the braided layers 3 are arranged and fit onto the outer walls of the support layers 5, and the braided layers 3 are braided with the same or different braiding yarns. Among which, the support layers 5 are made of flexible and deformable material; in this embodiment, such flexible and deformable material is polytetrafluoroethylene (PTFE), PTFE material has the characteristic of extremely low friction coefficient, therefore, the support layers 5 made of PTFE material are very smooth and conducive to arranging various functional tubes and wires in the support layer 5, such as device channel, transmission signal line and cord; the tubular structures of the support layers 5 can be made of suspended PTFE resin and integrally formed with an extruder, the thicknesses of the support layers 5 are 0.12 mm˜0.18 mm, and the bore diameters are 2.52 mm˜2.55 mm.

The connection tube 1 also consists of the sleeves 4 made of nylon elastomer material, the sleeves 4 are arranged and fit onto the outer walls of the braided layers 3; in this embodiment, the sleeves 4 are made of nylon elastomer material, their inner diameters gradually increase along the direction from the fixing ring 8 to the operation tube 2, the range of such inner diameters is 2.9 mm˜3.2 mm, and the wall thicknesses of the sleeves 4 are 0.1 mm˜0.15 mm.

As shown in FIG. 6, the reinforcing part 41 also made of nylon elastomer is arranged on one end of the sleeve 4 away from the operation tube 2, the stiffness of the reinforcing part 41 is between that of the flexible part 11 and the transition part 12; in this embodiment, the material of reinforcing part 41 is Pebax 5533 SA01 manufactured by French Arkema, and the stiffness is 55 HSD. The guide head 51 integrally formed with the same materials of the support layer 5 is arranged at the end of the support layer 5 of the fixing ring 8, the notch 511 used to connect the imaging device is arranged on the guide head 51 (FIGS. 4˜5); in this embodiment, the imaging device is a CMOS image sensor. The outer diameter of the guide head 51 is smaller than the inner diameter of the port of the support layer 5 with which the guide head 51 is connected, that is, the positioning step 52 is formed between the guide head 51 and the support, layer 5. The reinforcing coat 6 of the same shape with the guide head 51 is arranged outside the guide head 51, that is, the notch 511 is also arranged on the reinforcing coat 6, and one end of the reinforcing coat 6 is partially embedded in the reinforcing part 41. Since the imaging device often needs to be replaced and disassembled, the reinforcing coat 6 is made of stainless steel (preferably SS304V) to enhance the stiffness and prevent damages.

Since the fixing ring 8 runs deep into the human body, when changing imaging angle of the CMOS image sensor by changing bending degree of the connection tube 1, the fixing ring 8 is always bent first. The stiffness ranges of the flexible part 11, the transition part 12 and the handheld part 13 can be eventually increased through the sleeves 4 with different stiffness and the braided layers 3 with different flexibilities, the demand of bending the connection tube 1 in sequence from the fixing ring 8 to the operation tube 2 can be satisfied, and better flexibility, tracking, torsion control and bending accuracy of the connection tube 1 can be realized. In addition, during bending, the connection tube 1 provides high controllability and will not easily break.

Stiffness test method for different parts of the connection tube 1 is as follows:

Step I: Take same-length test samples in sequence from different parts of the connection tube 1 along the direction from the operation tube 2 to the fixing ring 8;

Step II: Fix both ends of a test sample, pull the middle part of the test sample with a tension meter, read tension value, the higher tension value is, the higher stiffness value of the corresponding part will be;

Step III: Adjust the material of the sleeve 4 and the braiding material, braiding method and warp & weft density of braiding of the braided layer 3 for each part according to the tension value of such part in order to gradually decrease the stiffness of parts along the direction from the fixing ring 8 to the operation tube 2;

Step IV: Conduct simulated bending test over the connection tube 1 with a human organ simulation model, eventually obtain the best stiffness values of different parts by judging whether bending degrees of such parts hinder the human organ simulation model, and whether normal operation requirements can be met.

The assembly process and working principle of the present invention are described below:

Firstly weld the traction wires 7 onto the lateral wall of the reinforcing coat 6 then apply AB glue onto the surfaces of the traction wires 7, align the traction wires 7 with the position of the notch 511 on the guide head 51, and align the traction wires 7 with the position of the wire trough 53, and sleeve the reinforcing coat 6 outside the guide head 51. When the reinforcing coat 6 butts with the positioning step 52, the assembly is finished, then place the traction wires 7 inside the wire troughs 53. Braid the braided layers 3 with braiding material from the fixing ring 8 and on outer walls of the support layers 5, meanwhile, input the support layers 5 covered with the braided layers 3 into the extruder, extrude the nylon elastomer material with the extruder and form the sleeves 4 to tightly wrap the support layers 5 and the braided layers 3, finally, cut off the nylon elastomer material from the reinforcing coat 6. Compared with the process in which the traction wires 7 are embedded into the support layers 5, then the braided layers 3 and the sleeves 4 are braided, and eventually welding is carried out, this process saves the time of assembling and disassembling the connection tube 1 into and from the mold and tooling for several times, therefore, processing steps are also simplified.

The above technical scheme only reflects the preferred technical scheme of the technical scheme of the present invention, the changes in certain aspects of the technical scheme made by those skilled in the art reflect the principle of the present invention and fall into the protection scope of the present invention.

1. An endoscope structure, which is featured by, consisting of a fixing ring, a connection tube and an operation tube connected in sequence, among which, the connection tube consists of braided layers, the connection tube, can be bent, and the stiffness of the braided layers of the connection tube is inversely proportional to the maximum bending angle of the connection tube;
The structure also consists of traction wires, the traction wires are fixed onto the fixing ring by the operation tube through the connection tube, and the connection tube will be bent by pulling the traction wires at the end of the operation tube.

2. An endoscope structure as recited in claim 1, which is featured by: the connection tube is a hollow tube composed of several layers of materials, the innermost layer of the connection tube is support layer, the support layers are integrally formed, the braided layers are arranged and fit onto the outer walls of the support layers, and the braided layers are braided with the same or different braiding yarns

3. An endoscope structure as recited in claim 2, which is featured by: the outer diameter of the connection tube is bigger than that of the fixing ring, a positioning step is formed at the junction between the guide head and the support layer, and the end of the reinforcing coat facing the connection tube contacts with the positioning step.

4. An endoscope structure as recited in claim 2, which is featured by: a guide groove corresponding to the traction wires is arranged on the outer lateral walls of the support layers, the traction wires are placed and can slide freely in the guide groove, and the guide groove is symmetrically arranged along the central axes of the support layers.

5. An endoscope structure as recited in claim 2, which is featured by: the connection tube consists of at least two connected parts with different stiffness, the part close to the operation tube is stiffer than the part close to the fixing ring, the stiffness of the parts can be controlled through changing the stiffness of the braided layers inside such parts, the support layers in different parts are made of the same flexible and deformable material, and the flexible and deformable material can be PTFE.

6. An endoscope structure as recited in claim 1, which is featured by: the number of the traction wires is at least two, one ends of the traction wires are fixed onto the lateral wall of the fixing ring, and the other ends extend outside the end of the operation tube along the wall of the connection tube, when one of the traction wires is pulled, the connection tube will bend toward the direction of the bended traction wire.

7. An endoscope structure as recited in claim 1, which is featured by: the connection tube also consists of sleeves made of nylon elastomer material, and the sleeves are arranged and fit onto the outer walls of the braided layer.

8. An endoscope structure as recited in claim 1, which is featured by: a guide head used to connect with the camera lens is arranged at the end of the fixing ring, and a notch convenient for fixed installation of the camera lens is arranged on the guide head.

9. An endoscope structure as recited in claim 8, which is featured by: the notch is preferably a U-shaped notch, a reinforcing coat of the same shape with the guide head is sleeved and arranged outside the guide head to enhance the firmness of the guide head.

10. An endoscope structure as recited in claim 7, which is featured by: a reinforcing se part ction is arranged at one end of the reinforcing coat close to the sleeve, the reinforcing coat is partially embedded in the reinforcing part.
